(19) United States
(12) Patent Application Publication
(10) Pub. No.: US 2024/0066279 A1
(43) Pub. Date: Feb. 29, 2024

(54) CATHETER TO PATIENT LINE CONNECTION ASSIST DEVICE

(71) Applicants: Baxter International Inc., Deerfield, IL (US); Baxter Healthcare SA, Glattpark (Opfikon) (CH)

(72) Inventors: Anoop Thirumattathil ASHOKAN, Bangalore (IN); Shashanka Belur Shanthakumar JAIN, Bangalore (IN)

(21) Appl. No.: 18/269,527

(22) PCT Filed: Dec. 14, 2021
(86) PCT No.: PCT/US2021/063273

§ 371 Date: Jun. 23, 2023

(30) Foreign Application Priority Data
Dec. 24, 2020 (IN) 202041056329

(51) Int. Cl. A61M 39/10; A61M 39/18

(52) U.S. Cl. CPC A61M 39/1011; A61M 39/18; A61M 2039/1066

(57) ABSTRACT

A connection assist device, which secures a connection of a catheter connector end to a patient line connector end, is provided. In one embodiment, the assist device includes a patient line shield, a catheter shield, and a minicap catcher. Each shield and catcher includes a first half and a second half. The patient line shied houses the patient line connector end. The catheter shield houses the catheter connector end. The minicap catcher is sized to receive a minicap initially protecting the catheter connector end. During use, the minicap catcher is inserted into the catheter shield and rotated to remove the minicap, after which the minicap connector is removed. The patient line shield is then inserted into the catheter shield housing to connect the catheter connector end to the patient line connector end.

### PRIORITY CLAIM

This application is a national stage under 35 USC 371 of PCT/US2021/063273, filed Dec. 14, 2021, which application claims priority to and the benefit of IN application 202041056329, filed on Dec. 24, 2020, entitled “Catheter to Patient Line Connection Assist Device”, the entirety of which is herein incorporated by reference.

### BACKGROUND

The present disclosure relates to medical fluid delivery and more specifically to medical fluid connections having reduced opportunity for contamination.

Peritoneal dialysis (“PD”) is a type of dialysis therapy used commonly to treat loss of kidney function. PD removes waste toxins and excess water from the patient by infusing a PD treatment fluid infused into a patient's peritoneal cavity via a catheter. The PD fluid contacts the peritoneal membrane of the peritoneal cavity. Waste, toxins and excess water pass from the patient's bloodstream, through the peritoneal membrane, and into the dialysis fluid via osmosis, i.e., an osmotic gradient occurs across the membrane. Used dialysis fluid is drained from the patient, removing waste, toxins and excess water from the patient. The above cycle may be repeated.

Two types of PD therapies are continuous ambulatory peritoneal dialysis (“CAPD”) and automated peritoneal dialysis (“APD”). CAPD is a manual dialysis treatment. The patient manually connects an implanted catheter to a drain, allowing used dialysis fluid, which had been previously infused into the patient in an earlier exchange, to drain from the patient's peritoneal cavity. The patient then connects the catheter to a bag of fresh dialysis fluid to infuse fresh dialysis fluid through the catheter and into the patient. The patient disconnects the catheter from the fresh dialysis bag and allows the dialysis fluid to dwell within the patient's peritoneal cavity, wherein the transfer of waste, toxins and excess water takes place as described herein. After the dwell period, the patient repeats the manual dialysis procedure, for example, four times per day, each of the procedures lasting about roughly an hour.

APD is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to sources or bags of fresh dialysis solution, and to a fluid drain. APD machines pump fresh dialysis solution from the sources or bags, through the catheter, into the patient's peritoneal cavity, and allow the dialysis fluid to dwell within the patient's peritoneal cavity for a period of time, causing the transfer of waste, toxins and excess water to take place. The APD machines then pump used dialysis fluid from the patient's peritoneal cavity to drain. As with the manual process, several drain, fill and dwell cycles occur during APD. A “last fill” may also occur at the end of both CAPD and APD, which remains in the peritoneal cavity of the patient until the next treatment or intermediate dialysis fluid exchange. The first cycle of the next treatment may accordingly begin with a drain of the previous last fill.

Catheters used with a PD machine typically have a connector end that attaches to a connector end of a patient line. The catheter can be provided in a variety of shapes and sizes, and typically includes a male luer connector. The patient line includes a patient line connector having a female luer, which connects to the male luer connector of the catheter.

The exposed end of a catheter connector is typically protected by a minicap when the catheter is not connected to the PD machine. To use the PD machine, the minicap is removed to expose the male catheter connector end that is inserted into the female connector end of the patient line. Separately, the female connector of the patient line is typically protected by a cap prior to treatment using the PD machine. The cap is removed in order to expose the female patient line connector that receives the male connector of the catheter.

To use the PD machine, the patient removes the minicap from the catheter connector and the cap from the patient line connector, and then inserts the catheter connector into the patient line connector. However, exposing the female and male connectors to ambient air even for a short amount of time creates a risk for the connectors to become contaminated, in particular through accidental touch. Such contamination can lead to peritoneal infections and other discomfort.

A need therefore exists for a device that minimizes the risk or prevents contamination of the connection of catheter and patient line connectors during the connection process.

### SUMMARY

The present disclosure includes a device that enables a connection between a catheter (including an indwelling tube that extends into the patient's peritoneal cavity) and a patient line running to a peritoneal dialysis treatment machine (for automated peritoneal dialysis (“APD”)) or to a tubing set (for continuous ambulatory peritoneal dialysis (“CAPD”)). More specifically, the present disclosure includes a device that protects the connector ends of the catheter and the patient line from unnecessary touch, thereby minimizing the risk of contamination.

In particular, a catheter to patient line connection assist device (“assist device”) that enables the connection of the catheter and the patient line while minimizing the risk of contamination is provided herein. The assist device enables the patient to secure the catheter to the patient line while minimizing the patient's contact at the juncture of the components. The assist device is configured to be compatible with any industrial catheter male luer connector and any industrial patient line female luer connector.

In one embodiment, the assist device includes a minicap catcher, a catheter shield, and a patient line shield. The three components are used sequentially in the process provided for a secure catheter to patient line connection. In an embodiment, the minicap catcher is first positioned about and engages the minicap on the catheter connector end, and then a catheter shield is positioned about the minicap catcher. The minicap catcher is rotated relative to the catheter shield to release the minicap from the catheter connector end. The minicap catcher is then withdrawn from the catheter shield, thereby removing the minicap and leaving the catheter connector end available for mating with the patient line connector end.

Separately, a patient line shield is first positioned about the patient line connector end, after which the cap is removed from the connector end. The patient line shield is then inserted into the catheter shield in order to connect the catheter connector end and the patient line connector end, thereby enabling the patient to use the PD machine.

The minicap catcher may be generally cylindrical and may include first and second clamshell halves that open rotatably about at least one hinge. The halves rotate about the hinge and join together when the minicap catcher is closed about the minicap of the catheter connector end.

Each clamshell half includes a minicap housing portion and an extension portion. An interior wall of the minicap housing portion is shaped to complement the external surface of a minicap, which has a cylindrical body having one or more planar surfaces in one embodiment. Planar surfaces on the interior wall allow the minicap catcher to engage with and rotate the minicap about the connector end of the catheter. Rotation of the minicap catcher within the catheter shield causes rotation of the minicap about the threaded male luer of the connector end.

The catheter shield may be generally cylindrical and may include first and second clamshell halves that open rotatably about at least one hinge. The halves rotate about the hinge and join together when the catheter shield is closed about the minicap catcher.

Each clamshell half includes a catheter housing portion and a minicap catcher housing portion. An interior wall of the catheter housing portion has a diameter that is sized to house any suitable industrial catheter. An interior wall of the minicap catcher housing portion is sized to receive the minicap catcher.

The extension portion of the minicap catcher extends outwardly from the minicap catcher housing portion of the catheter shield when the catheter shield is positioned about the minicap catcher. The user then grasps the extension portion of the minicap catcher and rotates it relative to the catheter shield. Rotation and withdrawal of the minicap catcher relative to and from the catheter shield causes rotation and withdrawal of the minicap relative to and from the connector end of the catheter, thereby exposing the connector end of the catheter within the minicap catcher housing portion of the catheter shield. The connector end of the catheter is now ready for connection to the patient line.

The patient line shield may be generally cylindrical and may include first and second clamshell halves that open rotatably about at least one hinge. Each clamshell half rotates about the hinge and joins with the other clamshell half when the patient line shield is closed about the cap.

Each clamshell half may include a patient line housing portion and a luer housing portion. An interior wall of the patient line housing portion has a diameter that is sized to house any suitable industrial patient line. An interior wall of the luer housing portion is sized to encompass the connector end of the patient line and the cap positioned thereon. The interior wall is also sized to allow for insertion of the male luer connector end of the catheter into the female luer connector end of the patient line.

The catheter shield, the patient line shield, and the minicap catcher may be reused after cleaning. The minicap and the patient line are disposable in one embodiment. An ultraviolet flash may be provided with the assist device to disinfect the minicap and patient line connection area as well.

In light of the disclosure set forth herein, and without limiting the disclosure in any way, in a first aspect, which may be combined with any other aspect or portion thereof described herein, a connection assist device for connecting a catheter connector end to a patient line connector end, the assist device comprising: a patient line shield including a first patient line half open and closeable relative to a second patient line half, the patient line shield including: a first patient line portion configured to house a portion of a patient line and a second patient line portion configured to house the patient line connector end; and a catheter shield including a first catheter shield half open and closeable relative to a second catheter shield half, the catheter shield including: a first catheter shield portion configured to house a portion of a patient catheter, and a second catheter shield portion configured to house the catheter connector end, the second catheter shield portion additionally sized to receive the second patient line portion of the patient line shield for connecting the catheter connector end to the patient line connector end.

In a second aspect, which may be combined with any other aspect or portion thereof described herein unless otherwise specified, the first catheter shield half is connected hingedly to the second catheter shield half.

In a third aspect, which may be combined with any other aspect or portion thereof described herein unless otherwise specified, the first patient line shield half is connected hingedly to the second patient line shield half.

In a fourth aspect, which may be combined with any other aspect or portion thereof described herein unless otherwise specified, the connection assist device includes a minicap catcher including a first minicap catcher half open and closeable relative to a second minicap catcher half, the minicap catcher including: a first minicap catcher portion sized to receive a minicap protecting the catheter connector end, and a second minicap catcher portion providing an extension surface, wherein the second catheter shield portion is sized to receive the first minicap catcher portion of the minicap catcher.

In a fifth aspect, which may be combined with any other aspect or portion thereof described herein unless otherwise specified, an inner wall of the first minicap catcher portion defines a cavity configured to engage the minicap.

In a sixth aspect, which may be combined with any other aspect or portion thereof described herein unless otherwise specified, at least one of the patient line shield, the catheter shield or the minicap catcher is at least partially cylindrical.

In a seventh aspect, which may be combined with any other aspect or portion thereof described herein unless otherwise specified, the second minicap catcher portion extends from the catheter shield when the second minicap catcher portion is inserted into the second catheter shield portion.

In an eighth aspect, which may be combined with any other aspect or portion thereof described herein unless otherwise specified, A connection assist device for connecting a catheter connector end to a patient line connector end, the assist device comprising: a minicap catcher including a first minicap catcher half open and closeable relative to a second minicap catcher half, the minicap catcher including: a first minicap catcher portion sized to receive a minicap on the catheter connector end; and a second minicap catcher portion providing a user gripping surface; a patient line shield including a first patient line half open and closeable relative to a second patient line half, the patient line shield including: a first patient line portion configured to house a portion of the patient line, and a second patient line portion configured to house the patient line connector end; and a catheter shield including a first catheter shield half open and closeable relative to a second catheter shield half, the catheter shield including a first catheter shield portion configured to house a portion of a patient catheter, and a second catheter shield portion configured to house the catheter connector end, the second catheter shield portion additionally sized to receive the first minicap catcher portion of the minicap catcher and the second patient line portion of the patient line shield.

In a ninth aspect, which may be combined with any other aspect or portion thereof described herein unless otherwise specified, a method of removing a minicap from a catheter connector end of a patient catheter and connecting the catheter connector end to a patient line connector end, the method comprises: locating a minicap catcher about the catheter connector end to engage the minicap protecting the catheter connector end; engaging a catheter shield about the minicap catcher, such that a first catheter shield portion engages a portion of the patient catheter, and a second catheter shield portion houses the minicap catcher; and rotating the minicap catcher relative to the catheter shield to remove the minicap from the catheter connector end; removing the minicap catcher from the catheter shield; engaging a patient line shield about the patient line connector end, such that: a first patient line portion engages a portion of the patient line, and a second patient line portion engages the patient line connector end; and inserting the second patient line portion into the second catheter shield portion such that the patient line connector end connects to the catheter connector end.

In a tenth aspect, which may be combined with any other aspect or portion thereof described herein unless otherwise specified, engaging the patient line shield includes clamshelling the patient line shield about the patient line connector end.

In an eleventh aspect, which may be combined with any other aspect or portion thereof described herein unless otherwise specified, inserting the second patient line portion into the second catheter shield portion includes threadingly connecting the patient line connector end connects to the catheter connector end.

In a twelfth aspect, any of the features, functionality and alternatives described in connection with any one or more of FIGS. 1 to 17 may be combined with any of the features, functionality and alternatives described in connection with any other of FIGS. 1 to 17.

It is accordingly an advantage of the present disclosure to connect a catheter connector end to a patient line connector end in a touch-free manner to increase safety during a CAPD or ADP treatment, which may be performed at home.

Additional features and advantages are described herein, and will be apparent from the following Detailed Description and the figures.

### BRIEF DESCRIPTION OF THE FIGURES

FIG. 1 is a perspective view of a distal end (with respect to an APD machine or cycler or to a CAPD disposable set) of a patient line having a patient line connector including a female luer, and the extension tube of a catheter including a male luer connector.

FIG. 2 is a perspective view of the assist device of the present disclosure connecting a connector end of a patient line to a connector end of a patient catheter.

FIG. 3 is a perspective view of an opened catheter shield of the assist device of the present disclosure.

FIG. 4 is a perspective view of an opened patient line shield of the assist device of the present disclosure.

FIG. 5 is a perspective view of an opened minicap catcher of the assist device of the present disclosure.

FIG. 6 is a perspective view illustrating a portion of the process for enclosing a patient catheter connector end of a patient transfer set within a minicap catcher of the assist device.

FIG. 7 is a perspective view illustrating a part of the process for enclosing a patient catheter connector end of a patient transfer set and minicap catcher within a catheter shield of the assist device.

FIG. 8 is a perspective view illustrating a part of the process for rotating the minicap catcher within the catheter shield.

FIG. 9 is a perspective view illustrating a part of the process for withdrawing the minicap catcher from the catheter shield, thereby removing the minicap from the catheter connector end.

FIG. 10 is a perspective view illustrating a part of the process for enclosing a cap on a patient line connector end within a patient line shield of the assist device.

FIG. 11 is a perspective view illustrating a part of the process for removing the cap from the patient line connector end within the patient line shield.

FIG. 12 is a perspective view illustrating a part of the process for inserting the patient line shield into the catheter shield.

FIG. 13 is a perspective view illustrating a part of the process for inserting the catheter connector end into the patient line connector end within the catheter and patient line shields.

FIG. 14 is an elevation, sectioned view illustrating the minicap catcher positioned about the minicap at the catheter connector end of the patient's transfer set.

FIG. 15 is an elevation, sectioned view illustrating the catheter shield positioned about the minicap catcher at the catheter connector end.

FIG. 16 is an elevation, sectioned view illustrating the catheter shield on the catheter connector end after the minicap catcher and minicap are removed.

FIG. 17 is an elevation, section view illustrating the catheter connector end and the patient line connector end secured together within the patient line shield and the catheter shield.

### DETAILED DESCRIPTION

Any of the components described herein, including components for assist device 20 and any components made of soft or hard materials, may be made of any suitable synthetic material or plastic, e.g., polyvinylchloride (“PVC”) or a non-PVC material, such as polyethylene (“PE”), polyurethane (“PU”) or polycarbonate (“PC”), and combinations thereof. Such materials may be molded or extruded as desired.

Referring now to the drawings, an assist device 20 discussed herein enables the patient to connect a connector end 2 of a peritoneal dialysis (“PD”) patient catheter 4 (device gaining access to a patient's peritoneal cavity) to a connector end 6 of a patient line 8 (running to an automated peritoneal dialysis (“APD”) cycler or to a continuous ambulatory peritoneal dialysis (“CAPD”) set) while protecting the connector ends 2, 6 from unnecessary touches by the patient or caregiver, thereby minimizing the risk of contamination. The assist device 20 is compatible with any industrial catheter male luer connector and any industrial patient line female luer connector, for example.

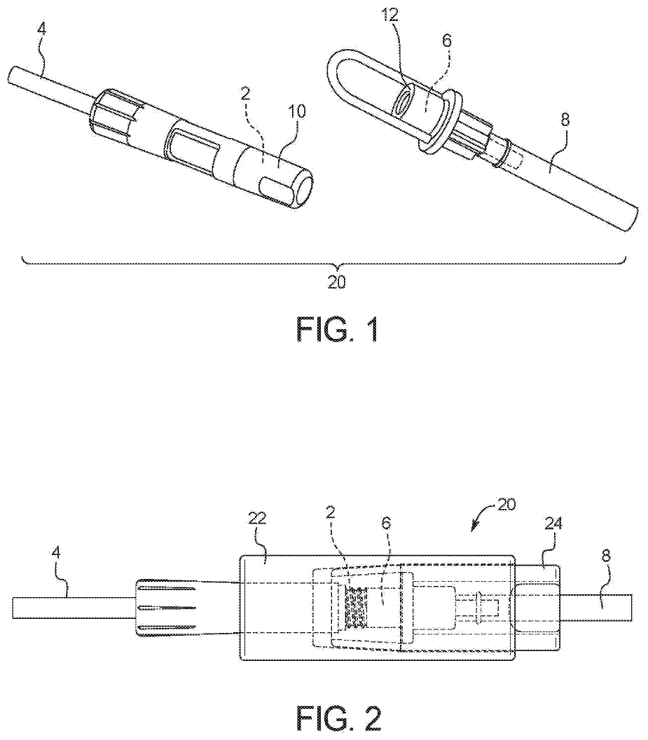

FIG. 1 shows an example of connector end 2 of patient catheter 4 and a connector end 6 of a patient line 8. While the connecter ends 2, 6 may be provided in a variety of shapes and sizes, connector end 2 is a male luer connector and connector end 6 is a female luer connector in one preferred embodiment. In the illustrated embodiment, the male connector end 2 of patient catheter 4 extends to a transfer set, which is capped via a minicap 10 during periods of non-use, e.g., prior to treatment. The transfer set is in one embodiment a MiniSet™ manufactured by the Baxter International Inc. Although the Mini Set™ is one suitable embodiment of the transfer set or catheter device of the present disclosure, assist device 20 can operate with any type of catheter device that couples to a tube or catheter inserted into the patient's peritoneal cavity. The female connector end 6 of the patient line 8 is secured within a cap 12 prior to use with the APD machine or CAPD set.

For treatment, the female connector end 6 of the patient line 8 is connected to the male connector end 2 of patient catheter 4 as shown in FIG. 2. The male luer connector end 2 of patient catheter 4 is externally threaded, while the female luer connector end 6 of the patient line 8 is housed in an internally threaded cavity that engages with the external threading of the male luer connector end of the patient catheter.

FIG. 2 illustrates an exemplary embodiment of a catheter shield 22 and a patient line shield 24 of the assist device 20 of the present disclosure placed about male and female connector ends 2, 6 of patient catheter 4 and the patient line 8, respectively, as the connector ends 2, 6 are secured together. The catheter shield 22 houses and makes available the connector end 2 of patient catheter 4 for insertion into the connector 6 of the patient line 8. The patient line shield 24 similarly houses and makes available the connector end 6 of the patient line 8 for receipt of the connector 2 of patient catheter 4.

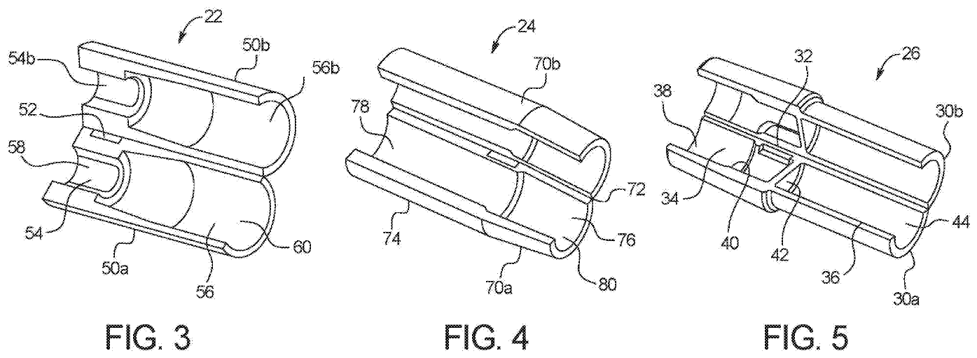

Referring to FIGS. 3 to 5, assist device 20 of the present disclosure may include three primary components, namely, catheter shield 22 and patient line shield 24 described above, and additionally a minicap catcher 26 that removes a minicap 10 (FIGS. 1 and 3) initially covering and protecting connector end 2 of patient catheter 4. FIGS. 3 to 13 demonstrate the steps for using the assist 20 to connect the catheter and patient lines 4, 8.

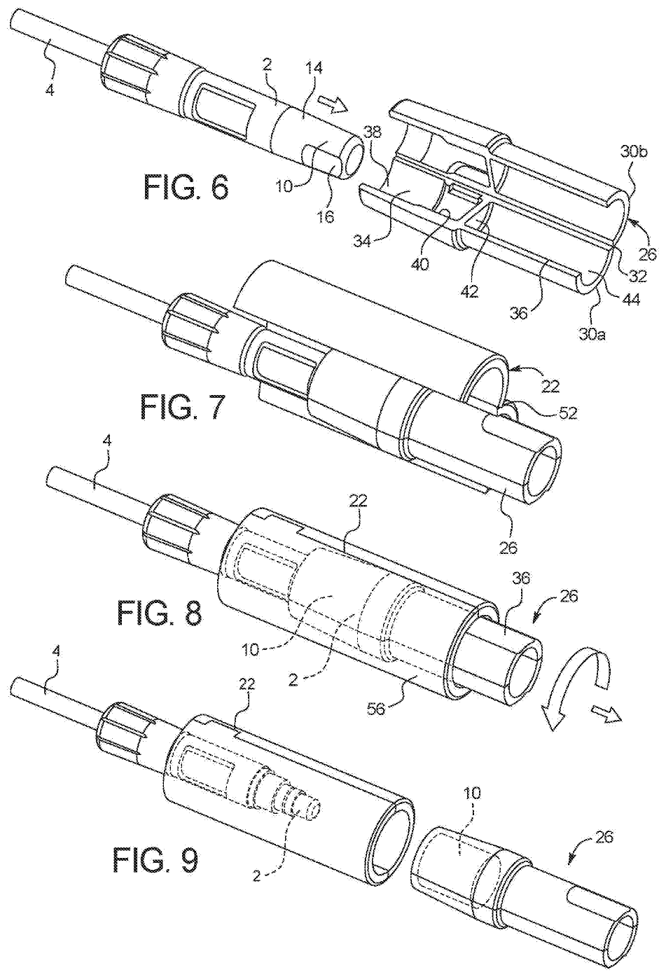

In particular, FIGS. 5 and 6 illustrate that minicap catcher 26 is in one embodiment cylindrical and includes first and second clamshell halves 30a, 30b that open rotatably about at least one hinge 32 (see FIG. 3). It should be appreciated that in various embodiments, the minicap catcher 26 may include a single hinge or multiple hinges. Halves 30a, 30b of the minicap catcher 26 may alternatively be free from each other.

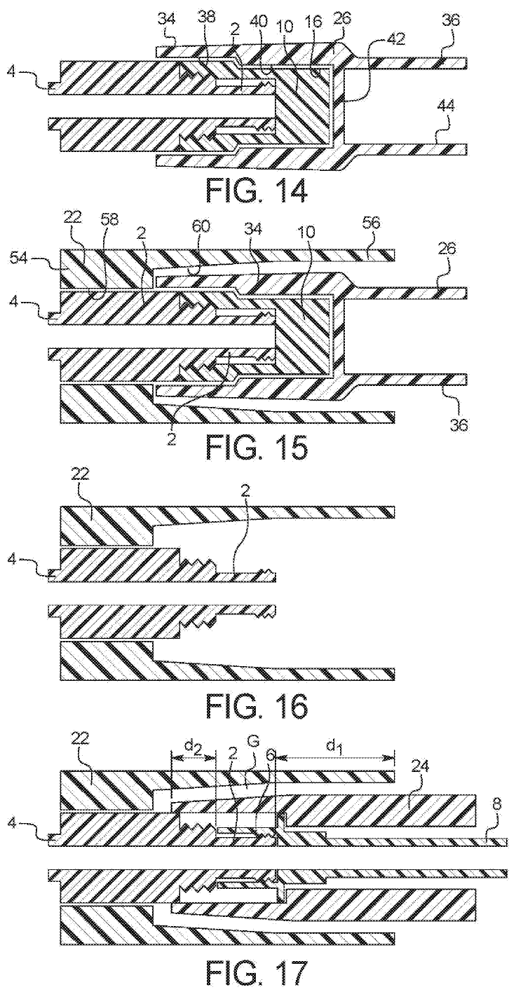

As shown in FIGS. 5 and 6, each clamshell half 30a, 30b includes a minicap housing portion 34 and an extension portion 36. The halves 30a, 30b rotate about the hinge 32 and join together when the minicap catcher 26 is closed about the minicap 10 of the catheter connector end 2 as shown in FIGS. 7 and 14. In one example, the clamshell halves 30a, 30b are held together by a closing clip that snap-fits together. It should be appreciated that in various embodiments, the minicap catcher 26 may include a single closing clip, another desirable number of closing clips, or no closing clips to hold halves 30a, 30b when closed together. Closing clips may alternatively or additionally include snap-fitting elements or a loop and pile (Velcro™) tape releasable fastener.

An interior wall 38 of the minicap housing portion 34 is shaped to complement the external surface of a minicap 10, which has a cylindrical body 14 with one or more planar surfaces 16 having a knurled finger grip. Matching planar surfaces 40 on the interior wall 38 allow the minicap catcher 26 to engage with the planar surfaces 16 of the minicap 10 and rotate the minicap 10 about the connector end 2 of patient catheter 4.

The planar surfaces 40 are adjacent to a transverse internal wall 42 within the minicap catcher 26 that separates the minicap housing portion 34 and the extension portion 36. The length of the minicap housing portion 34 is dimensioned to capture the full length of the minicap 10 in the illustrated embodiment. However, it can be appreciated that the minicap housing portion 34 (including halves 30a, 30b of portion 34 in FIG. 5) may have a length that is greater than or less than the full length of the minicap 10.

The extension portion 36 of the minicap catcher 26 provides a handle or gripping area that extends outwardly from the catheter shield 22 when enclosed therewithin. The internal surface 44 of the extension portion 36 is cylindrical and hollow in the illustrated embodiment, although the extension portion 36 (including portion halves 36a and 36b in FIG. 4A) may be solid or include ribbing or other internal structures in other embodiments.

Referring to FIGS. 3 and 7, the catheter shield 22 is in one embodiment cylindrical and includes first and second clamshell halves 50a, 50b that open rotatably about at least one hinge 52. It should be appreciated that in various embodiments, the catheter shield 22 may include a single hinge or multiple hinges. Halves 50a, 50b of the catheter shield 22 may alternatively be free from each other.

As shown in FIG. 3, each clamshell half 50a, 50b includes a catheter housing portion 54 and a minicap catcher housing portion 56. The halves 50a, 50b rotate about the hinge 52 and join together when the catheter shield 22 is closed about the minicap catcher 24 as shown in FIG. 7. It should be appreciated that in various embodiments, the catheter shield 22 may include a single closing clip, another desirable number of closing clips, or no closing clips to hold clamshell halves 50a, 50b together when closed against each other. Closing clips may alternatively or additionally include snap-fitting elements or a loop and pile (Velcro) tape releasable fastener.

As illustrated in FIGS. 3 and 15, an interior wall 58 of the catheter housing portion 54 of catheter shield 22 has a diameter that is sized to house any suitable industrial catheter, such as patient catheter 4 illustrated in FIG. 1. An interior wall 60 of the minicap catcher housing portion 56 is sized to house the minicap catcher 26.

Referring to FIGS. 8 and 9, the extension portion 36 of the minicap catcher 26 extends outwardly from the minicap catcher housing portion 56 of the catheter shield 22 when the minicap catcher 26 is captured within the catheter shield 22. The user then grasps the minicap catcher 26 and rotates it relative to the catheter shield 22. Rotation and withdrawal of the minicap catcher 26 relative to and from the catheter shield 22 causes rotation and withdrawal of the minicap 10 relative to and from the connector end 2 of patient catheter 4, thereby exposing the connector end 2 of the catheter within the minicap catcher housing portion 56 of the catheter shield 22 as seen in FIGS. 9 and 16. The connector end 2 of patient catheter 4 is now ready for connection to the patient line 8.

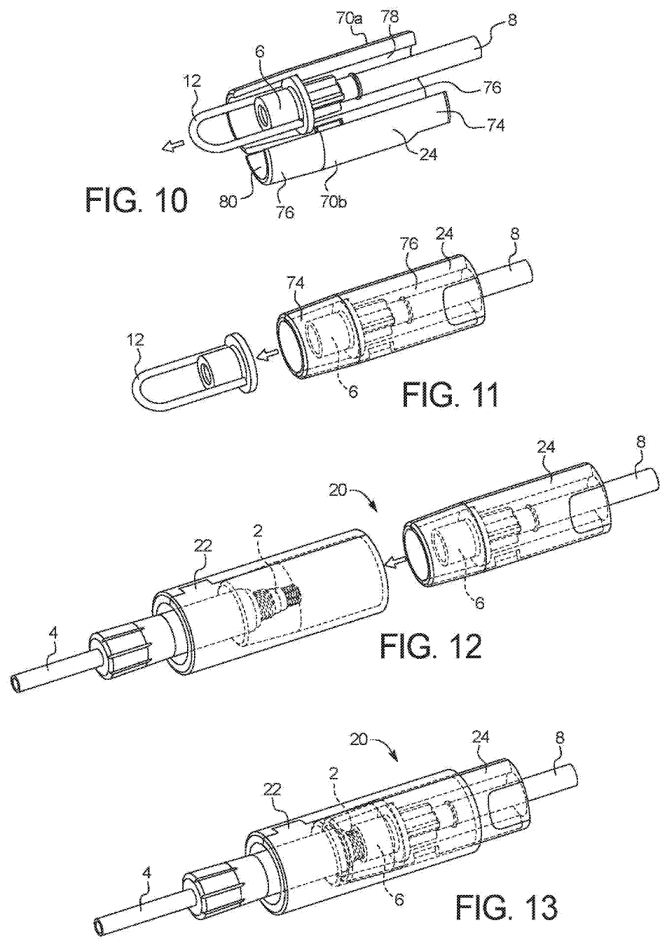

Referring to FIGS. 4 and 10, the patient line shield 24 is in one embodiment cylindrical and includes first and second clamshell halves 70a, 70b that open rotatably about at least one hinge 72. It should be appreciated that in various embodiments, the patient line shield may include a single hinge or multiple hinges. Clamshell halves 70a, 70b of the patient line shield may alternatively be free from each other.

As shown in FIG. 4, each clamshell half 70a, 70b includes a patient line housing portion 74 and a luer housing portion 76. Each half 70a, 70b rotates about the hinge 72 and, wherein halves 70a, 70b join together when the patient line shield 24 is closed about the cap 12 as shown in FIG. 10. It should be appreciated that in various embodiments, the patient line shield 24 may include a single closing clip, another desirable number of closing clips, or no closing clips to hold clamshell halves 70a, 70b together when closed against each other. Closing clips may alternatively or additionally include snap-fitting elements or a loop and pile (Velcro) tape releasable fastener.

Referring again to FIG. 10, an interior wall 78 of the patient line housing portion 74 has a diameter that is sized to house any suitable industrial patient line, such as patient line 8 illustrated in FIGS. 1 and 10. An interior wall 80 of the luer housing portion 76 is sized to house the connector end 6 of the patient line 8 and the cap 12 positioned thereon. Interior wall 80 of patient line shield 24 is also sized to allow for insertion of the male luer connector end 2 of patient catheter 4 into the female luer connector end 6 of the patient line 8, as seen in FIG. 13.

As shown in FIGS. 10 and 11, the user positions the patient line shield 24 about the cap 12 protecting the connector end 6 of the patient line 8 and then removes the cap 12 from the connector end 6, leaving the connector end 6 available for mating with the connector end 2 of the catheter line 4.

Referring to FIGS. 12, 13, and 17, the user inserts the patient line shield 24 into the catheter shield 22 and rotates the connector ends 2, 6 relative to one another to make and ensure a tight fluid connection, such as a threaded luer connection, between connector end 2 and connector end 6. The user may then remove reusable patient line shield 24 and reusable catheter shield 22, leaving an exposed but connected patient catheter 4 and patient line 8.

In the embodiment illustrated in FIG. 17, catheter shield 22 and patient line shield 24 extend past connector ends 2, 6, respectively, distances d1 (for shield 22) and d2 (for shield 24), wherein the distances are suitable to prevent or make it very difficult for a patient or caregiver's finger to extend into the shields far enough to make contact with connector ends 2, 6. Because the inner diameter of catheter shield 22 is larger and thus more accessible than the smaller diameter of patient line shield 24, distance d1 of catheter shield 22 may be larger than distance d2 of patient line shield 24 to provide adequate protection. The distances d1 and d2 may be sized so that the user's thumb does not reach the connector end 2, 6 when pressed against the outer end of the catheter shield 22 and patient line shield 24, respectively. Additionally, the shields 22, 24 are sized such that an annular gap G between the outer surface of the patient line shield 24 and the inner surface of the catheter shield 22 is minimized to easily guide the components together and avoid any potential contact and related touch contamination between the patient line shield 24 and the catheter connector end 2.

FIGS. 14 to 17 illustrate sectioned views of the components of assist device 20 at distinct points during the method of use. In FIG. 14, the minicap catcher 26 is secured about the minicap 10 at the connector end 2 of the catheter line 4, and wherein minicap 10 is connected threadingly to the connector end 2. In other embodiments, the minicap 10 may be connected to the connector end 2 through a friction lock instead of a threaded connection. FIG. 15 illustrates the catheter shield 22 positioned about the minicap catcher 26 at the connector end 2. FIG. 16 illustrates the connector end 2 of the catheter line 4 within the patient shield 22 once minicap 10 and minicap catcher 26 have been rotated and withdrawn. Here, the open end of patient shield 22 is shown protecting the exposed connector end 2 of the catheter line 4. Finally, FIG. 17 illustrates the connector end 2 of patient catheter 4 and the connector end 6 of the patient line 8 connected, e.g., threadingly connected, within the patient line shield 24, which is housed within the catheter shield 22. Notably, the inner diameter of the mating end of patient line shield 24 may be larger than a largest diameter of the connector end 2 of the catheter line 4, so that the mating end of patient line shield 24 can extend over the largest diameter of the connector end 2 when the connector end 2 is filly threaded to connector end 6 of the patient line 8.

In some embodiments, the catheter shield 22, the patient line shield 24, and the minicap catcher 26 of the assist device 20 may be reused after cleaning. Minicap 10 is disposable in one embodiment. An ultraviolet lamp may be provided with assist device 20 to disinfect the connection area while the connected catheter and patient line ends 2, 6 are housed within the catheter and patient line shields 22, 24. The UV lamp may be configured to provide UV light flashes to disinfect mated ends 2, 6. In some embodiments, the UV lamp may include lights or light-emitting diodes (“LEDs”) housed within the patient line shield 24 directed toward the connector ends 2, 6. The UV lights may be activated when the catheter connector end 2 is detected by a sensor within the patient line connector end 6. The UV lights may also be manually operated. In another embodiment, a UV flash is applied to the components 22, 24, 26 once removed from the catheter line 4 and patient line 8. Further, the UV lamps may for example be battery powered. The UV lights/LEDs and battery may be housed within the catheter shield 22 and/or the patient line shield 24.

During treatment, the user may leave the catheter shield 22 and patient line shield 24 in position on the catheter and patient line ends 2, 6. The user may alternatively remove the catheter shield 22 and patient line shield 24 during treatment and reposition them about the catheter and patient line ends 2, 6 for disconnection.

To disconnect the patient from patient line 8 after treatment, the user prepares for the disconnection by first readying a new minicap 10 within a minicap catcher 26, wherein the new minicap 10 is to be applied promptly to the connector end 2 of the catheter line 4 once disconnected from the connector end 6 of the patient line 8. In one embodiment, the user positions the minicap 10 within minicap housing portion 34 of minicap catcher 26 and closes the minicap catcher 26 around the new minicap 10. In some embodiments, the minicap 10 is prepackaged within a minicap catcher 26.

If the catheter shield 22 and patient line shield 24 are removed during use, the user then reapplies patient line shield 24 about patient line 8 including its connector end 6 and thereafter reapplies catheter shield 22 about (i) connector end 2 of the catheter line 4 and (ii) about the reapplied patient line shield 24.

The user then (i) separates (unthreads) the connector end 6 of the patient line 8 from the connector end 2 of patient catheter 4, (ii) twists patient line shield 24 and patient line 8 relative to patient connector 4 and catheter shield 22 and pulls patient line shield 24 from catheter shield 22, and (iii) inserts the minicap housing portion 34 of the minicap catcher 26 into the minicap catcher housing portion 56 of the catheter shield 22. The user presses the new minicap 10 into place on the connector end 2 of patient catheter 4 and rotates the minicap catcher 26 so that the new minicap 10 translates onto the connector end 2 of patient catheter 4 via a threaded connection. The clamshell halves 50a, 50b of the catheter shield 22 are then unlocked and the catheter shield 22 is removed. The minicap catcher 26 is then pulled off of the new minicap 10, leaving the new minicap 10 in place protecting the connector end 2 of patient catheter 4. The user removes the patient line shield 24 from patient line 8 and discards the patient line along with the reminder of a disposable set providing the patient line.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A connection assist device for connecting a catheter connector end to a patient line connector end, the assist device comprising:
a patient line shield including a first patient line half open and closeable relative to a second patient line half, the patient line shield including:
a first patient line portion configured to house a portion of a patient line, and
a second patient line portion configured to house the patient line connector end; and
a catheter shield including a first catheter shield half open and closeable relative to a second catheter shield half, the catheter shield including:
a first catheter shield portion configured to house a portion of a patient catheter, and
a second catheter shield portion configured to house the catheter connector end, the second catheter shield portion additionally sized to receive the second patient line portion of the patient line shield for connecting the catheter connector end to the patient line connector end.

2. The connection assist device of claim 1, wherein the first catheter shield half is connected hingedly to the second catheter shield half.

3. The connection assist device of claim 1, wherein the first patient line shield half is connected hingedly to the second patient line shield half.

4. The connection assist device of claim 1, further comprising a minicap catcher including a first minicap catcher half open and closeable relative to a second minicap catcher half, the minicap catcher including:
a first minicap catcher portion sized to receive a minicap protecting the catheter connector end, and
a second minicap catcher portion providing an extension surface, wherein the second catheter shield portion is sized to receive the first minicap catcher portion of the minicap catcher.

5. The connection assist device of claim 4, wherein an inner wall of the first minicap catcher portion defines a cavity configured to engage the minicap.

6. The connection assist device of claim 4, wherein at least one of the patient line shield, the catheter shield or the minicap catcher is at least partially cylindrical.

7. The connection assist device of claim 4, wherein the second minicap catcher portion extends from the catheter shield when the second minicap catcher portion is inserted into the second catheter shield portion.

8. A connection assist device for connecting a catheter connector end to a patient line connector end, the assist device comprising:
a minicap catcher including a first minicap catcher half open and closeable relative to a second minicap catcher half, the minicap catcher including:
a first minicap catcher portion sized to receive a minicap on the catheter connector end; and
a second minicap catcher portion providing a user gripping surface;
a patient line shield including a first patient line half open and closeable relative to a second patient line half, the patient line shield including:
a first patient line portion configured to house a portion of the patient line, and
a second patient line portion configured to house the patient line connector end; and
a catheter shield including a first catheter shield half open and closeable relative to a second catheter shield half, the catheter shield including
a first catheter shield portion configured to house a portion of a patient catheter, and
a second catheter shield portion configured to house the catheter connector end, the second catheter shield portion additionally sized to receive the first minicap catcher portion of the minicap catcher and the second patient line portion of the patient line shield.

9. A method of removing a minicap from a catheter connector end of a patient catheter and connecting the catheter connector end to a patient line connector end, the method comprising:
locating a minicap catcher about the catheter connector end to engage the minicap protecting the catheter connector end;
engaging a catheter shield about the minicap catcher, such that
a first catheter shield portion engages a portion of the patient catheter, and
a second catheter shield portion houses the minicap catcher; and
rotating the minicap catcher relative to the catheter shield to remove the minicap from the catheter connector end;
removing the minicap catcher from the catheter shield;
engaging a patient line shield about the patient line connector end, such that:
a first patient line portion engages a portion of the patient line, and
a second patient line portion engages the patient line connector end; and
inserting the second patient line portion into the second catheter shield portion such that the patient line connector end connects to the catheter connector end.

10. The method of claim 9, wherein engaging the patient line shield includes clamshelling the patient line shield about the patient line connector end.

11. The method of claim 9, wherein inserting the second patient line portion into the second catheter shield portion includes threadingly connecting the patient line connector end connects to the catheter connector end.
